(19) United States
(12) Patent Application Publication
(10) Pub. No.: US 2010/0261690 A1
(43) Pub. Date: Oct. 14, 2010

(54) NOVEL COMBINATIONS

(75) Inventors: Frank Burkamp, Sodertalje (SE); Peter Hansen, Lund (SE)

Correspondence Address: ASTRAZENECA R&D BOSTON, 35 GATEHOUSE DRIVE, WALTHAM, MA, 02451-1215, US

(73) Assignee: ASTRAZENECA R&D, Sodertalje (SE)

(21) Appl. No.: 12/749,888

(22) Filed: Mar. 30, 2010

Related U.S. Application Data
(60) Provisional application No. 61/166,310, filed on Apr. 3, 2009.

(51) Int. Cl. A61K 31/58; A61P 11/00

(52) U.S. Cl. 514/171

(57) ABSTRACT

The invention provides a pharmaceutical product comprising, in combination, (1) a named glucocorticosteroid receptor agonist and (2) a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist, and the use of said product in treating respiratory diseases.

This application claims the benefit of U.S. Provisional application No. 61/166,310, filed Apr. 3, 2009, the disclosure of which is incorporated by reference herein in its entirety.

The present invention relates to a combination of two or more pharmaceutically active substances for use in the treatment of respiratory diseases (for example chronic obstructive pulmonary disease (COPD) or asthma).

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of is great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term that refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi that causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease that affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include corticosteroids. Corticosteroids (also known as glucocorticosteroids or glucocorticoids) are potent anti-inflammatory agents. Whilst their exact mechanism of action is not clear, the end result of corticosteroid treatment is a decrease in the number, activity and movement of inflammatory cells into the bronchial submucosa, leading to decreased airway responsiveness. Corticosteroids may also cause reduced shedding of bronchial epithelial lining, vascular permeability, and mucus secretion. Whilst corticosteroid treatment can yield important benefits, the efficacy of these agents is often far from satisfactory, particularly in COPD. Moreover, whilst the use of steroids may lead to therapeutic effects, it is desirable to be able to use steroids in low doses to minimise the occurrence and severity of undesirable side effects that may be associated with regular administration. Recent studies have also highlighted the problem of the acquisition of steroid resistance amongst patients suffering from respiratory diseases. For example, cigarette smokers with asthma have been found to be insensitive to short term inhaled corticosteroid therapy, but the disparity of the response between smokers and non-smokers appears to be reduced with high dose inhaled corticosteroid (Tomlinson et al., Thorax 2005; 60:282-287).

A further class of therapeutic agent used in the treatment of respiratory diseases are bronchodilators. Bronchodilators may be used to alleviate symptoms of respiratory diseases by relaxing the bronchial smooth muscles, reducing airway obstruction, reducing lung hyperinflation and decreasing shortness of breath. Types of bronchodilators in clinical use include β₂ adrenoceptor agonists, muscarinic receptor antagonists and methylxanthines. Bronchodilators are prescribed mainly for symptomatic relief and they are not considered to alter the natural history of respiratory diseases.

Combination products comprising a β₂ adrenoceptor agonist and a corticosteroid are available. One such product is a combination of budesonide and formoterol fumarate dihydrate (marketed by AstraZeneca under the trade mark Symbicort®), which has proven to be effective in controlling asthma and COPD, and improving quality of life in many patients.

In view of the complexity of respiratory diseases such as asthma and COPD, it is unlikely that any one mediator can satisfactorily treat a respiratory disease alone. Whilst the known combination treatments using a β₂ adrenoceptor agonist and a corticosteroid deliver significant patient benefits, there remains a medical need for new therapies against respiratory diseases such as asthma and COPD, in particular for therapies with disease modifying potential.

In accordance with the present invention there is provided a pharmaceutical product comprising a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate; a second active ingredient selected from a β₂ adrenoreceptor agonist, a dual β₂ adrenoreceptor agonist/M₃ receptor antagonist (hereinafter referred to as a “MABA compound”), a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor (GR receptor) agonist; and optionally one or more pharmaceutically acceptable excipients.

Thus, in this embodiment the first and second active ingredients are in admixture.

The compound, (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, is disclosed in our co-pending International Patent Application No. PCT/GB2008/050890 (WO 2009/044200) and has glucocorticosteroid receptor agonist activity.

The invention also provides a pharmaceutical product comprising a preparation of a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, and a preparation of a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor (GR receptor) agonist, wherein the preparations are for simultaneous, sequential or separate administration to a patient in need thereof.

The present invention further provides a kit comprising a preparation of a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, and a preparation of a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor (GR receptor) agonist, and instructions for the simultaneous, sequential or separate administration of the preparations to a patient in need thereof.

By “simultaneous” is meant that the preparations of the first and second active ingredients are administered at the same time. By “sequential” is meant that the preparations of the first and second active ingredients are administered, in any order, one immediately after the other. They still have the desired effect if they are administered separately, but when administered in this manner they are generally administered less than 4 hours apart, conveniently less than two hours apart, more conveniently less than 30 minutes apart and most conveniently less than 20 minutes apart, for example less than 10 minutes but not one immediately after the other.

The first active ingredient, (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, may exist in solvated, for example hydrated, as well as unsolvated forms, and the present invention encompasses all such solvated forms.

A β₂-adrenoreceptor agonist is any compound or substance capable of stimulating the β₂-receptors and acting as a bronchodilator. In the context of the present specification, unless otherwise stated, any reference to a β₂-adrenoreceptor agonist includes an active salt, solvate or derivative that may be formed from said β₂-adrenoreceptor agonist or any enantiomer or mixture thereof. Examples of possible salts or derivatives of a β₂-adrenoreceptor agonist are

(1) acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, and

(2) pharmaceutically acceptable esters (e.g. C₁-C₆ alkyl esters). The β₂-adrenoreceptor agonists may also be in the form of solvates, e.g. hydrates.

Examples of β₂-adrenoreceptor agonists that may be used in the pharmaceutical product according to the invention include:

metaproterenol,

isoproterenol,

isoprenaline,

albuterol,

salbutamol (e.g. as sulphate),

formoterol (e.g. as fumarate or fumarate dihydrate),

salmeterol (e.g. as xinafoate),

terbutaline,

orciprenaline,

bitolterol (e.g. as mesylate),

pirbuterol or

indacaterol.

In one embodiment of the invention, the β₂-adrenoreceptor agonist is a long-acting β₂-adrenoreceptor agonist (i.e. a (β₂-adrenoreceptor agonist with activity that persists for more than 24 hours), examples of which include: salmeterol (e.g. as xinafoate),

formoterol (e.g. as fumarate or fumarate dihydrate),

is bambuterol (e.g. as hydrochloride),

carmoterol (TA 2005, chemically identified as [R-(R*,R*)]-8-hydroxy-5-[1-hydroxy-2-[[2-(4-methoxy-phenyl)-1-methylethyl]-amino]ethyl]-2(1H)-quinolone monohydrochloride, also identified by Chemical Abstract Service Registry Number 137888-11-0 and disclosed in U.S. Pat. No. 4,579,854),

a benzothiazolone as disclosed in WO 2005/074924, or WO 2006/056741 (for example, 7-[(R)-2-((1S,2S)-2-Benzyloxy-cyclopentylamino)-1-hydroxyethyl]-4-hydroxy-3H-benzothiazol-2-one),

an aryl aniline as disclosed in WO 2003/042164 or WO 2006/133942 (for example, N-[2-[4-[(3-phenyl-4-methoxyphenyl)amino]phenyl]ethyl]-(R)-2-hydroxy-2-(8-hydroxy-1,2-dihydro-2-oxoquinolin-5-yl)ethylamine),

compounds disclosed in WO 2006/07489 (for example, 5-[(R)-2-(2-{4-[4-(2-amino-2-methyl-propoxy)-phenylamino]-phenyl}-ethylamino)-1-hydroxyethyl]-8-hydroxy-1H-quinolin-2-one),

a formanilide as disclosed in WO 2004/011416, WO 2005/030678, or WO 2006/066907 (for example, N-(2-[4-((R)-2-hydroxy-2-phenylethylamino)phenyl]ethyl)-(R)-2-hydroxy-2-(3-formamido-4-hydroxyphenyl)ethylamine),

compounds disclosed in WO 2005/121065 (for example, 8-hydroxy-5-[(1R)-1-hydroxy-2-[6-(phenethylamino)hexylamino]ethyl]-1H-quinolin-2-one),

compounds disclosed in WO 2003/024439 (for example, (1R)-4-[2-[6-[2-[(2,6-dichlorophenyl)methoxy]ethoxy]hexylamino]-1-hydroxyethyl]-2-(hydroxymethyl)phenol),

compounds disclosed in WO 2004/037773 (for example, 4-[(1R)-2-[6-[4-(3-cyclopentylsulfonylphenyl)butoxy]hexylamino]-1-hydroxyethyl]-2-(hydroxymethyl)phenol),

a benzenesulfonamide derivative as disclosed in WO 2002/066422 (for example, 3-(4-{[6-({(2R)-2-hydroxy-2-[4-hydroxy-3-(hydroxy-methyl)phenyl]ethyl}amino)-hexyl]oxy}butyl)benzenesulfonamide),

a formanilide disclosed in WO 2002/076933 (for example, 3-(4-{[6-({(2R)-2-[3-(formylamino)-4-hydroxyphenyl]-2-hydroxyethyl}amino)hexyl]oxy}-butyl)-benzenesulfonamide),

a compound GSK159797, GSK159802, GSK597901, GSK642444 or GSK678007, an indole derivative as disclosed in WO 2004/032921 (for example, N-[(2,6-dimethoxyphenyl)methyl]-5-[2-[[2-hydroxy-2-[4-hydroxy-3-(hydroxymethyl)phenyl]ethyl]amino]propyl]-1H-indole-2-carboxamide),

compounds disclosed in WO 2006/051375 (for example, N-(1-adamantyl)-2-[3-[(2R)-2-[[(2R)-2-hydroxy-2-[4-hydroxy-3-(hydroxymethyl)phenyl]ethyl]amino]propyl]phenyl]acetamide),

compounds disclosed in WO 2008/017637 (for example 8-[(1R)-2-[[4-[3-(4-chlorophenyl)-5-methyl-1,2,4-triazol-1-yl]-2-methylbutan-2-yl]amino]-1-hydroxyethyl]-6-hydroxy-4H-1,4-benzoxazin-3-one),

compounds disclosed in WO 2008/023003 (for example, N-[5-[(1R)-2-[[4-(4,4-diethyl-2-oxo-3,1-benzoxazin-1-yl)-2-methylbutan-2-yl]amino]-1-hydroxyethyl]-2-hydroxyphenyl]methanesulfonamide),

compounds disclosed in WO 2006/122788, and WO 2008/095720 (for example, 5-(2-{[6-(2,2-difluoro-2-phenylethoxy)hexyl]amino}-1-hydroxyethyl)-8-hydroxyquinolin-2(1H)-one), compounds disclosed in WO 2008/046598 (for example, 5-[(1R)-2-[2-[4-(2,2-difluoro-2-phenylethoxy)phenyl]ethylamino]-1-hydroxyethyl]-8-hydroxy-1H-quinolin-2-one), and

compounds disclosed in WO 2007/124898 (for example, 5-(2-[(6-(2-[(2,6-dichlorobenzyl)(methyl)amino]ethoxy)hexyl)amino]-1-hydroxyethyl)-8-hydroxyquinolin-2(1H)-one).

In another embodiment of the invention, the β₂-adrenoreceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide as disclosed in WO 2008/096111,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide as disclosed in WO 2008/096121,
7-[(1R)-2-({2-[(3-{[2-(2-Chlorophenyl)ethyl]amino}1 propyl)thio]ethyl}amino)-1-hydroxyethyl]-4-hydroxy-1,3-benzothiazol-2(3H)-one as outlined in WO 2008/104776,
4-Hydroxy-7-[1-hydroxy-2-(2-{3-[(2-methoxy-benzylamino)-methyl]-phenyl}-ethylamino)-ethyl]-3H-benzothiazol-2-one as disclosed in WO 2008/106016, or
N-Cyclohexyl-3-[2-(3-fluorophenyl)ethylamino]-N-[2-[2-(4-hydroxy-2-oxo-3H-1,3-benzothiazol-7-yl)ethylamino]ethyl]propanamide as disclosed in WO 2008/075026, is or a pharmaceutically acceptable salt of any one thereof.

In yet another embodiment of the invention, the β₂-adrenoreceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide,
4-Hydroxy-7-[1-hydroxy-2-(2-{3-[(2-methoxy-benzylamino)-methyl]-phenyl}-ethylamino)-ethyl]-3H-benzothiazol-2-one dihydrobromide, or
N-Cyclohexyl-3-[2-(3-fluorophenyl)ethylamino]-N-[2-[2-(4-hydroxy-2-oxo-3H-1,3-benzothiazol-7-yl)ethylamino]ethyl]propanamide di-D-mandelate salt.

A MABA compound is a compound having dual activity as both a muscarinic antagonist and as a β₂-adrenoreceptor agonist, examples of which are disclosed in WO 2004/089892, WO 2004/106333, US 2004/0167167, WO 2005/111004, WO 2005/051946, US 2005/0256114, WO 2006/023457, WO 2006/023460, US 2006/0223858, US 2006/0223859, WO 2007/107828, WO 2008/000483, U.S. Pat. No. 7,317,102 and WO 2008/041095.

Specific examples of MABA compounds include:
biphenyl-2-ylcarbamic acid 1-[2-(4-{[(R)-2-(3-formylamino-4-hydroxyphenyl)-2-hydroxyethylam-2,5-dimethylphenylcarbamoyl)ethyl]piperidin-4-yl ester,
succinic acid salt of biphenyl-2-ylcarbamic acid 1-[2-(2-chloro-4-{[(R)-2-hydroxy-2-(8-hydroxy-2-oxo-1,2-dihydroquinolin-5-yl)ethylmino]methyl}-5-methoxyphenylcarbamoyl)ethyl]piperidin-4-yl ester,
naphthalene-1,5-disulfonic acid salt of biphenyl-2-ylcarbamic acid 1-(9-[(R)-2-hydroxy-2-(8-hydroxy-2-oxo-1,2-dihydro-quinolin-5-yl)ethylamino]nonyl}piperidin-4-yl ester, and
N-{5-[(1R)-2-((2-[4-(2-{3-[(1R)-3-(diisopropylamino)-1-phenylpropyl)-4-hydroxyphenyl}ethoxy)-phenyl]ethyl}amino)-1-hydroxyethyl]-2-hydroxyphenyl}methanesulfonamide (optionally as the succinate salt).

Examples of muscarinic antagonists that may be used in the pharmaceutical product according to the invention include:
aclidinium bromide,
glycopyrrolate (such as R,R-, R,S-, S,R-, or S,S-glycopyrronium bromide),
oxitropium bromide,
pirenzepine,
telenzepine,
tiotropium bromide,
darotropium ((1R,3R,5S)-3-(2-cyano-2,2-diphenylethyl)-8,8-dimethyl-8-azoniabicyclo[3,2,1]octane bromide),
3(R)-(2-hydroxy-2,2-dithien-2-ylacetoxy)-1-(3-phenoxypropyl)-1-azoniabicyclo[2.2.2]octane bromide (see WO 01/04118),
3 (R)-1-phenethyl-3-(9H-xanthene-9-carbonyloxy)-1-azoniabicyclo[2.2.2]octane bromide,
(3R)-3-[(2S)-2-cyclopentyl-2-hydroxy-2-thien-2-ylacetoxy]-1-(2-phenoxyethyl)-1-azoniabicyclo[2.2.2]actane bromide (see WO 01/04118),
((R)-3-(1-phenyl-cycloheptanecarbonyloxy)-1-(pyridin-2-ylcarbamoylmethyl)-1-azonia-bicyclo[2.2.2]octane salt, e.g. bromide salt, as described in WO 2009/139708, and
quaternary ammonium salts such as [2-((S)-Cyclohexyl-hydroxy-phenyl-methyl)-oxazol-5-ylmethyl]-dimethyl-(3-phenoxy-propyl)-ammonium salt, [2-((R)-Cyclohexyl-hydroxy-phenyl-methyl)-oxazol-5-ylmethyl]-dimethyl-(3-phenoxy-propyl)-ammonium salt, [2-((R)-Cyclohexyl-hydroxy-phenyl-methyl)-oxazol-5-ylmethyl]-dimethyl-(2-phenethyloxy-ethyl)-ammonium salt, [2-((R)-Cyclohexyl-hydroxy-phenyl-methyl)-oxazol-5-ylmethyl]-[3-(3,4-dichloro-phenoxy)-propyl] dimethyl-ammonium salt, [2-((R)-Cyclohexyl-hydroxy-phenyl-methyl)-oxazol-5-ylmethyl]-[2-(3,4-dichloro-benzyloxy)-ethyl]-dimethyl-ammonium salt or [2-(4-Chloro-benzyloxy)-ethyl]-[2-((R)-Cyclohexyl-hydroxy-phenyl-methyl)-oxazol-5-ylmethyl]-dimethyl-ammonium salt, or (R)-1-[2-(4-Fluoro-phenyl)-ethyl]-3-((S)-2-phenyl-2-piperidin-1-yl-propionyloxy)-1-azonia-bicyclo[2.2.2]octane where the counter ion is, for example, chloride, bromide, sulfate, methanesulfonate, benzenesulfonate (besylate), toluenesulfonate (tosylate), napthalenebissulfonate (napadisylate), hemi-napthalene-bissulfonate (hemi-napadisylate), phosphate, acetate, citrate, lactate, tartrate, mesylate, maleate, fumarate or succinate.

p38 Kinase inhibitors are known, for example, from WO 2009/001132. One such compound described in WO 2009/001132 is N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide and pharmaceutically acceptable salts thereof.

A suitable salt of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide is, for example, a hydrochloride, hydrobromide, trifluoroacetate, sulphate, phosphate, acetate, fumarate, maleate, tartrate, lactate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate, 2-naphthalenesulphonate, 2,5-dichlorobenzenesulphonate, D-mandelate, L-mandelate, cinnamate, benzoate, adipate, esylate, malonate, mesitylate (2-mesitylenesulphonate), napsylate (2-naphthalenesulfonate), camsylate (camphor-10-sulphonate, for example (1S)-(+)-10-Camphorsulfonic acid salt), formate, glutamate, glutarate, glycolate, hippurate (2-(benzoylamino)acetate), orotate, xylate (p-xylene-2-sulphonate), pamoic (2,2′-dihydroxy-1,1′-dinaphthylmethane-3,3′-dicarboxylate), palmitate or furoate. It is to be understood for the avoidance of confusion that salts may exist in varying stoichiometries, for example, but not limited to, hemi-, mono-, and di-, and that the invention encompasses all such forms.

A neutrophil elastase inhibitor is, for example, 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide (WO 2007/129963).

Phosphodiesterase PDE4 inhibitors are known in the art and include, for example, 6-fluoro-N-((1s,4s)-4-(6-fluoro-2,4-dioxo-1-(4′-(piperazin-1-ylmethyl)-biphenyl-3-yl)-1,2-dihydropyrido[2,3-d]pyrimidin-3(4H)-yl)cyclohexyl)imidazo[1,2-a]pyridine-2-carboxamide (as disclosed in WO 2008/084223), or a pharmaceutically acceptable salt thereof, for example, a (1S)-(+)-10-camphorsulfonic acid or trihydrochloride salt; and 6-Fluoro-N-((1s,4s)-4-(6-fluoro-2,4-dioxo-1-(4′-(piperazin-1-ylmethyl)-biphenyl-3-yl)-1,2-dihydropyrido[2,3-d]pyrimidin-3(4H)-yl)cyclohexyl)imidazo[1,2-a]pyridine-2-carboxamide (as described in International Patent Application No. PCT/GB2008/000061), or a pharmaceutically acceptable salt thereof such as a (1S)-(+)-10-camphorsulfonic acid salt.

An IKK2 kinase inhibitor is, for example, 2-{[2-(2-Methylamino-pyrimidin-4-yl)-1H-indole-5-carbonyl]-amino}-3-(phenyl-pyridin-2-yl-amino)-propionic acid or a compound as disclosed in WO 01/58890, WO 03/010158, WO 03/010163, WO 04/063185 or WO 04/063186.

A non-steroidal glucocorticoid receptor (GR) agonist is, for example, a compound disclosed in WO 2008/076048, for example 2, 2,2-trifluoro-N-[(1R,2S)-1-[1-(4-fluorophenyl)indazol-5-yl]oxy-1-(3-methoxyphenyl)propan-2-yl]acetamide, N-[(1R,2S)-1-[1-(4-fluorophenyl)indazol-5-yl]oxy-1-(4-methylsulfonylphenyl)propan-2-yl]-2-hydroxy-acetamide, N-[(1R*,2S*)-1-[1-(4-fluorophenyl)indazol-5-yl]oxy-1-(6-methoxypyridin-3-yl)propan-2-yl]cyclopropanecarboxamide, (2S)-N-[(1R,2S)-1-[1-(4-fluorophenyl)indazol-5-yl]oxy-1-phenyl-propan-2-yl]-2-hydroxy-propanamide, 2,2,2-trifluoro-N-[(2S*,3S*)-3-[1-(4-fluorophenyl)indazol-5-yl]oxy-4-phenoxy-butan-2-yl]acetamide, N-[(1R,2S)-1-[1-(4-fluorophenyl)indazol-5-yl]oxy-1-(3-methoxyphenyl)propan-2-yl]-N-propan-2-yl-oxamide, or a pharmaceutically acceptable salt thereof.

In a preferred aspect of the invention, the second active ingredient is selected from:
N-[2-(diethylamino)ethyl]-N-(2-{[2-(4-hydroxy-2-oxo-2,3-dihydro-1,3-benzothiazol-7-yl)ethyl]amino}ethyl)-3-(2-naphthalen-1-ylethoxy)propanamide,
(3R)-1-[2-(4-fluorophenyl)ethyl]-3-{[(2S)-2-phenyl-2-piperidin-1-ylpropanoyl]oxy}-1-azoniabicyclo[2.2.2]octane,
(3R)-1-[2-oxo-2-(pyridin-2-ylamino)ethyl]-3-{[(1-phenylcycloheptyl)carbonyl]oxy}-1-azoniabicyclo[2.2.2]octane,
N-cyclopropyl-3-fluoro-4-methyl-5-{3-[(1-{2-[2-(methylamino)ethoxy]phenyl}cyclopropyl)amino]-2-oxopyrazin-1(2H)-yl}benzamide,
N-cyclohexyl-N-(2-{[2-(5-hydroxy-3-oxo-3,4-dihydro-2H-1,4-benzoxazin-8-yl)ethyl]amino}ethyl)-3-{2-[3-(1-methyl-1H-pyrazol-4-yl)phenyl]ethoxy} propanamide,
N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide,
   or a pharmaceutically acceptable salt thereof.

All the above second et seq active ingredients may be in the form of solvates, for example hydrates.

The active ingredients may be delivered to the lung and/or airways via oral administration in the form of a solution, suspension, aerosol or dry powder formulation. These dosage forms will usually include one or more pharmaceutically acceptable excipients which may be selected, for example, from adjuvants, carriers, binders, lubricants, diluents, stabilising agents, buffering agents, emulsifying agents, viscosity-regulating agents, surfactants, preservatives, flavourings or colorants. Examples of such excipients are described in the Handbook of Pharmaceutical Excipients (Fifth Edition, 2005, edited by Ray C. Rowe, Paul J. Sheskey and Sian C. Owen, published by the American Pharmaceutical Association and the Pharmaceutical Press). The active ingredients of the present invention may also be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. As will be understood by those skilled in the art, the most appropriate method of administering the active ingredients is dependent on a number of factors.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the first active ingredient is administered via inhalation. When administered via inhalation the dose of the first active ingredient will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the second active ingredient is administered by inhalation. When administered via inhalation the dose of the second active ingredient will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In another embodiment the present invention provides a pharmaceutical product wherein the molar ratio of first active ingredient to second active ingredient is from 1:1000 to 1000:1, such as from 1:100 to 100:1, for example from 1:50 to 50:1, for example 1:20 to 20:1.

In one preferred embodiment, the pharmaceutical product comprising a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate; a second active ingredient selected from a β₂ adrenoreceptor agonist, a dual (β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist; and optionally one or more pharmaceutically acceptable excipients, is formulated for inhaled administration.

In another preferred embodiment, the pharmaceutical product comprising a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate; a second active ingredient selected from a β₂ adrenoreceptor agonist, a dual (β₂ adrenoreceptor agonist/M₃ receptor antagonist or a muscarinic antagonist; and optionally one or more pharmaceutically acceptable excipients, is formulated for inhaled administration.

In still another preferred embodiment, the preparations of the first and second active ingredients for simultaneous, sequential or separate administration are each formulated for inhaled administration.

Administration by inhalation may be via the oral or nasal route using a pressurised metered dose inhaler (pMDI), a nebuliser or a dry powder inhaler.

If a pMDI is used, the first and/or second active ingredient(s) may be dispersed in a suitable propellant optionally together with an additional excipient such as an alcohol (e.g. ethanol), a surfactant, a lubricant or a stabilising agent. A suitable propellant includes a hydrocarbon, chlorofluorocarbon or a hydrofluoroalkane (e.g. heptafluoroalkane) propellant, or a mixture of any such propellants. Preferred propellants are P134a and P227, each of which may be used alone or in combination with other another propellant and/or surfactant and/or other excipient.

If a nebuliser is used, the first and/or second active ingredient(s) will typically be formulated as an aqueous suspension or, preferably, solution, with or without suitable pH and/or tonicity adjustment.

A dry powder inhaler may be used to administer the active ingredients, alone or in combination with a pharmaceutically acceptable carrier (such as lactose), in the latter case either as a finely divided powder or as an ordered mixture. The dry powder inhaler may be “passive” or breath-actuated, or “active” where the powder is dispersed by some mechanism other than the patient's inhalation, for instance, an internal supply of compressed air. At present, three types of passive dry powder inhalers are available: single-dose, multiple unit dose or multidose (reservoir) inhalers. In single-dose devices, individual doses are provided, is usually in capsules, and have to be loaded into the inhaler before use, examples of which include Spinhaler® (Aventis), Rotahaler® (GlaxoSmithKline), Aeroliser™ (Novartis), Inhalator® (Boehringer) and Eclipse (Aventis) devices. Multiple unit dose inhalers contain a number of individually packaged doses, either as multiple gelatine capsules or in blisters, examples of which include Diskhaler® (GlaxoSmithKline), Diskus® (GlaxoSmithKline), Aerohaler® (Boehringer) and Handihaler® (Boehringer) devices. In multidose devices, drug is stored in a bulk powder reservoir from which individual doses are metered, examples of which include Turbuhaler® (AstraZeneca), Easyhaler® (Orion), Novohzer® (ASTA Medica), Clickhaler® (Innovata Biomed) and Pulvinal® (Chiesi) devices.

Thus, the present invention further provides a dry powder inhaler, in particular a multiple unit dose dry powder inhaler, containing a pharmaceutical product as hereinbefore described.

The pharmaceutical product of the present invention may be used to treat diseases of the respiratory tract such as obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated to with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

Accordingly, the present invention further provides a pharmaceutical product as hereinbefore defined for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The present invention further provides the use of first and second active ingredients, wherein the first active ingredient is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate and the second active ingredient is a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist, in the manufacture of a medicament or pharmaceutical product for the treatment of a respiratory disease, in particular chronic obstructive pulmonary disease, asthma, rhinitis, emphysema or bronchitis.

In one embodiment, the present invention provides the use of first and second active ingredients, wherein the first active ingredient is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate and the second active ingredient is a β₂ adrenoreceptor agonist, a dual β₂ adrenoreceptor agonist/M₃ receptor antagonist or a muscarinic antagonist, in the manufacture of a medicament or pharmaceutical product for the treatment of a respiratory disease, in particular chronic obstructive pulmonary disease, asthma, rhinitis, emphysema or bronchitis.

The present invention still further provides a method of treating a respiratory disease which comprises simultaneously, sequentially or separately administering to a patient in need thereof:

(a) a therapeutically effective dose of a first active ingredient as defined above; and

(b) a therapeutically effective dose of a second active ingredient as defined above.

### EXAMPLE 1

### Inhibition of Lipopolysaccharride (LPS)-Induced TNFα Production in Human Peripheral Blood Mononuclear Cells

Human isolated peripheral blood mononuclear cells (PBMCs) were pre-incubated with a range of concentrations of the GR agonist (1R,3aS,3bS,10aR,10bS,11S,12aS)-1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate (Compound A), alone or in the presence of a range of concentrations of a second compound with a distinct pharmacological activity for 45 minutes at 37° C. After the pre-incubation period, the cells were then incubated with LPS (5 ng/mL) for 18 hr at 37° C. to induce TNFα production. The total assay volume was 200 μL. At the end of the incubation period, 10 μL of the culture supernatant diluted 1:5 was analysed to quantify the TNFα released using AlphaLISA (PerkinElmer). The fluorescence was detected on an EnVision Alphareader. Inhibition curves were fitted using a 4-parameter logistic equation in a non-linear curve fitting routine and activity was expressed as pIC50. In this series of experiments, the test of Compound A alone gave a pIC50 for inhibition of LPS-induced TNFα production from human PBMC of 9.15±0.09 (n=7 exp).

In the particular series of experiments described below, compound A was tested in combination with each of the Compounds B to J described in the following table. In the table the chemical structure of each of the exemplified compounds is depicted together with the chemical name used in the present specification to denote the compound parent structure.

| Com- | | | |
|---|---|---|---|
| pound | Chemical Structure | Mechanism | IUPAC Name ACD v10.06 |
| A | 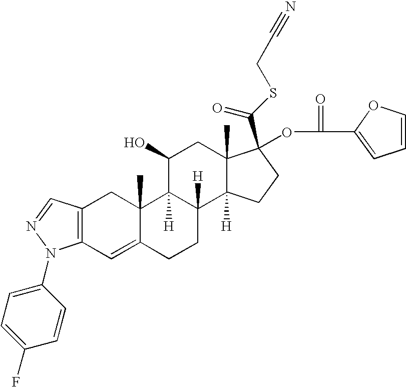 | GR agonist | (1R,3aS,3bS,10aR,10bS,11S,l2aS)-1- {[(cyanomethyl)sulfanyl]carbonyl}-7-(4- fluorophenyl)-11-hydroxy-10a,12a-dimethyl- 1,2,3,3a,3b,4,5,7,10,10a,10b,11,12,12a- tetradecahydrocyclopenta[5,6]naphtho[1,2- f]indazol-1-yl furan-2-carboxylate |
| B | 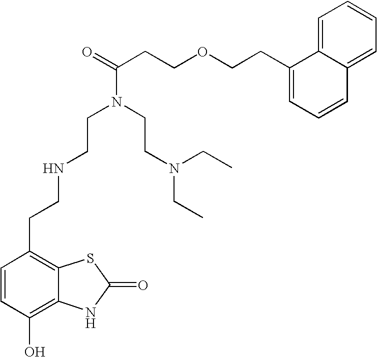 | LABA | N-[2-(diethylamino)ethyl]-N-(2-{[2-(4-hydroxy- 2-oxo-2,3-dihydro-1,3-benzothiazol-7- yl)ethyl]amino}ethyl)-3-(2-naphthalen-1- ylethoxy)propanamide |
| C | 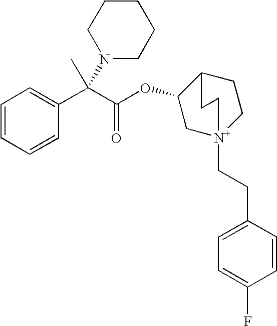 | LAMA | (3R)-1-[2-(4-fluorophenyl)ethyl]-3-{[(2S)-2- phenyl-2-piperidin-1-ylpropanoyl]oxy}-1- azoniabicyclo[2.2.2]octane |
| D | 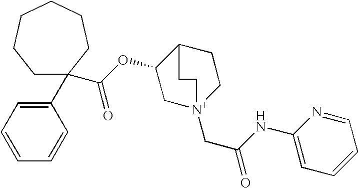 | LAMA | (3R)-1-[2-oxo-2-(pyridin-2-ylamino)ethyl]-3- {[(1-phenylcycloheptyl)carbonyl]oxy}-1- azoniabicyclo[2.2.2]octane |
| E | 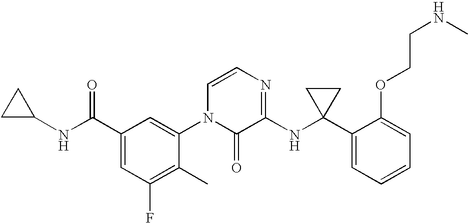 | p38 | N-cyclopropyl-3-fluoro-4-methyl-5-{3-[(1-{2- [2-(methylamino)ethoxy]phenyl}cyclopropyl) amino]-2-oxopyrazin-1(2H)-yl}benzamide |
| F | 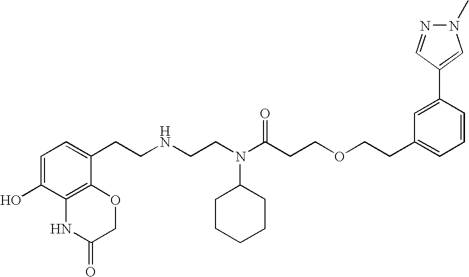 | LABA | N-cyclohexyl-N-(2-{[2-(5-hydroxy-3-oxo-3,4- dihydro-2H-1,4-benzoxazin-8- yl)ethyl]amino}ethyl)-3-{2-[3-(1-methyl-1H- pyrazol-4-yl)phenyl]ethoxy}propanamide |
| G | | LABA | N-Cyclohexyl-N³-[2-(3-fluorophenyl)ethyl]-N- (2-{[2-(4-hydroxy-2-oxo-2,3-dihydro-1,3- benzothiazol-7-yl)ethyl]amino}ethyl)-β- alaninamide |
| H | 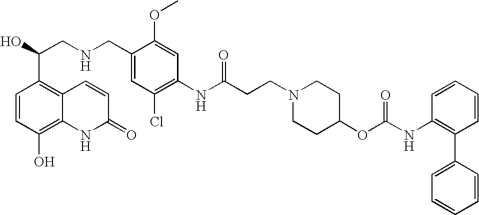 | MABA | 1-(3-{[2-chloro-4-({[(2R)-2-hydroxy-2-(8- hydroxy-2-oxo-1,2-dihydroquinolin-5- yl)ethyl]amino}methyl)-5- methoxyphenyl]amino}-3-oxopropyl)piperidin- 4-yl biphenyl-2-ylcarbamate |
| I | 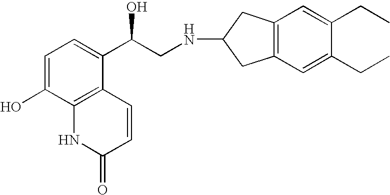 | LABA | 5-{(1R)-2-[(5,6-diethyl-2,3-dihydro-1H-inden- 2-yl)amino]-1-hydroxyethyl}-8- hydroxyquinolin-2(1H)-one |
| J | 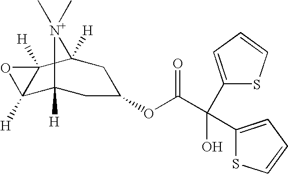 | LAMA | (1R,2R,4S,5S,7S)-7{[hydroxy(dithiophen-2- yl)acetyl]oxy}-9,9-dimethyl-3-oxa-9- azoniatricyclo[3.3.1.0~2,4~]nonane |

The pIC₅₀ and maximal inhibition achieved for combinations of Compound A with each of Compounds B to J are shown are in Tables 1 to 9 below. In each table, the data represents the mean of two separate experiments using PBMC from healthy blood donors (n=2).

TABLE 1
Compound A in combination with Compound B

| | Concentration | pIC50 | % inhibition @ |
|---|---|---|---|
| Compound | (μM) | compound A | 1000 nM |
| Compound B | 1 | 9.1 | 83.8 |
| Compound B | 0.1 | 9.0 | 82.5 |
| Compound B | 0.01 | 9.1 | 81.6 |
| Compound B | 0.001 | 9.6 | 76.6 |
| Compound B | 0.0001 | 9.2 | 74.8 |
| Compound B | 0.00001 | 9.2 | 74.2 |
| Compound B | 0.000001 | 9.1 | 76.5 |
| Compound B | 0 | 9.2 | 76.7 |

TABLE 2
Compound A in combination with Compound C

| | Concentration | pIC50 | % inhibition @ |
|---|---|---|---|
| Compound | (μM) | compound A | 1000 nM |
| Compound C | 1 | 9.4 | 72.1 |
| Compound C | 0.1 | 9.1 | 75.3 |
| Compound C | 0.01 | 9.2 | 73.2 |
| Compound C | 0.001 | 9.7 | 73.2 |
| Compound C | 0.0001 | 9.3 | 70.9 |
| Compound C | 0.00001 | 9.3 | 72.2 |
| Compound C | 0.000001 | 9.1 | 73.4 |
| Compound C | 0 | 9.2 | 73.3 |

TABLE 3
Compound A in combination with Compound D

| | Concentration | pIC50 | % inhibition @ |
|---|---|---|---|
| Compound | (μM) | compound A | 1000 nM |
| Compound D | 1 | 9.2 | 71.9 |
| Compound D | 0.1 | 9.2 | 71.6 |
| Compound D | 0.01 | 9.3 | 71.2 |
| Compound D | 0.001 | 9.6 | 70.5 |
| Compound D | 0.0001 | 9.2 | 71.2 |
| Compound D | 0.00001 | 9.4 | 71 |
| Compound D | 0.000001 | 9.1 | 71.1 |
| Compound D | 0 | 9.3 | 71.5 |

TABLE 4
Compound A in combination with Compound E

| | Concentration | pIC50 | % inhibition @ |
|---|---|---|---|
| Compound | (μM) | compound A | 1000 nM |
| Compound E | 1 | 9.2 | 99.4 |
| Compound E | 0.1 | 9.6 | 99.1 |
| Compound E | 0.01 | 9.1 | 96.7 |
| Compound E | 0.001 | 9.2 | 80.2 |
| Compound E | 0.0001 | 9.1 | 73.6 |
| Compound E | 0.00001 | 9.3 | 72.4 |
| Compound E | 0.000001 | 9.1 | 73.4 |
| Compound E | 0 | 9.1 | 74.3 |

TABLE 5
Compound A in combination with Compound F

| | Concentration | pIC50 | % inhibition @ |
|---|---|---|---|
| Compound | (μM) | compound A | 1000 nM |
| Compound F | 1 | 9.0 | 78.6 |
| Compound F | 0.1 | 9.0 | 77.8 |
| Compound F | 0.01 | 9.2 | 78 |
| Compound F | 0.001 | 9.4 | 74.6 |
| Compound F | 0.0001 | 9.2 | 68.4 |
| Compound F | 0.00001 | 9.2 | 68.3 |
| Compound F | 0.000001 | 9.3 | 67.9 |
| Compound F | 0 | 9.3 | 68.9 |

TABLE 6
Compound A in combination with Compound G

| | Concentration | pIC50 | % inhibition @ |
|---|---|---|---|
| Compound | (μM) | compound A | 1000 nM |
| Compound G | 1 | 8.7 | 79.5 |
| Compound G | 0.1 | 8.8 | 79.6 |
| Compound G | 0.01 | 8.8 | 78.0 |
| Compound G | 0.001 | 9.0 | 75.4 |
| Compound G | 0.0001 | 9.1 | 70.9 |
| Compound G | 0.00001 | 9.0 | 69.9 |
| Compound G | 0.000001 | 8.9 | 69.8 |
| Compound G | 0 | 9.2 | 71.8 |

TABLE 7
Compound A in combination with Compound H

| | Concentration | pIC50 | % inhibition @ |
|---|---|---|---|
| Compound | (μM) | compound A | 1000 nM |
| Compound H | 1 | 8.5 | 74.6 |
| Compound H | 0.1 | 8.4 | 74.9 |
| Compound H | 0.01 | 8.5 | 73.4 |
| Compound H | 0.001 | 8.6 | 67.9 |
| Compound H | 0.0001 | 8.6 | 62.4 |
| Compound H | 0.00001 | 8.5 | 59.9 |
| Compound H | 0.000001 | 8.4 | 62.8 |
| Compound H | 0 | 8.9 | 63.0 |

TABLE 8
Compound A in combination with Compound I

| | Concentration | pIC50 | % inhibition @ |
|---|---|---|---|
| Compound | (μM) | compound A | 1000 nM |
| Compound I | 1 | 8.4 | 72.7 |
| Compound I | 0.1 | 8.3 | 72.5 |
| Compound I | 0.01 | 8.3 | 71.3 |
| Compound I | 0.001 | 8.3 | 64.1 |
| Compound I | 0.0001 | 8.6 | 56.5 |
| Compound I | 0.00001 | 8.7 | 59.5 |
| Compound I | 0.000001 | 8.3 | 59.4 |
| Compound I | 0 | 8.6 | 59.6 |

TABLE 9
Compound A in combination with Compound J

| | Concentration | pIC50 | % inhibition @ |
|---|---|---|---|
| Compound | (μM) | compound A | 1000 nM |
| Compound J | 1 | 8.7 | 53.9 |
| Compound J | 0.1 | 8.6 | 51.9 |
| Compound J | 0.01 | 8.5 | 51.0 |
| Compound J | 0.001 | 8.5 | 53.9 |
| Compound J | 0.0001 | 8.4 | 49.2 |
| Compound J | 0.00001 | 8.4 | 51.7 |
| Compound J | 0.000001 | 8.1 | 57.4 |
| Compound J | 0 | 8.4 | 51.8 |

1. A pharmaceutical product comprising a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate; a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist; and optionally one or more pharmaceutically acceptable excipients.

2. A pharmaceutical product according to claim 1 which is in a form suitable for administration by inhalation.

3. A dry powder inhaler containing a pharmaceutical product as claimed in claim 1 or claim 2.

4. A dry powder inhaler according to claim 3 which is a multiple unit dose dry powder inhaler.

5. A pharmaceutical product comprising a preparation of a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, and a preparation of a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist, wherein the preparations are for simultaneous, sequential or separate administration to a patient in need thereof.

6. A pharmaceutical product according to claim 5, wherein the preparations of the first and second active ingredients are each in a form suitable for administration by inhalation.

7. A pharmaceutical product according to claim 1 in which the second active ingredient is selected from:
N-[2-(diethylamino)ethyl]-N-(2-{[2-(4-hydroxy-2-oxo-2,3-dihydro-1,3-benzothiazol-7-yl)ethyl]amino}ethyl)-3-(2-naphthalen-1-ylethoxy)propanamide,
(3R)-1-[2-(4-fluorophenyl)ethyl]-3-{[(2S)-2-phenyl-2-piperidin-1-ylpropanoyl]oxy}-1-azoniabicyclo[2.2.2]octane,
(3R)-1-[2-oxo-2-(pyridin-2-ylamino)ethyl]-3-{[(1-phenylcycloheptyl)carbonyl]oxy}-1-azoniabicyclo[2.2.2]octane,
N-cyclopropyl-3-fluoro-4-methyl-5-{3-[(1-{2-[2-(methylamino)ethoxy]phenyl}cyclopropyl)amino]-2-oxopyrazin-1(2H)-yl}benzamide,
N-cyclohexyl-N-(2-{[2-(5-hydroxy-3-oxo-3,4-dihydro-2H-1,4-benzoxazin-8-yl)ethyl]amino}ethyl)-3-{2-[3-(1-methyl-1H-pyrazol-4-yl)phenyl]ethoxy}propanamide,
N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide,
or a pharmaceutically acceptable salt thereof.

8. A method of treating a respiratory disease which comprises simultaneously, sequentially or separately administering to a patient in need thereof:
(a) a therapeutically effective dose of a first active ingredient being (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate; and
(b) a therapeutically effective dose of a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist.

9. A method according to claim 8 wherein the second active ingredient is selected from:
N-[2-(diethylamino)ethyl]-N-(2-{[2-(4-hydroxy-2-oxo-2,3-dihydro-1,3-benzothiazol-7-yl)ethyl]amino}ethyl)-3-(2-naphthalen-1-ylethoxy)propanamide,
(3R)-1-[2-(4-fluorophenyl)ethyl]-3-{[(2S)-2-phenyl-2-piperidin-1-ylpropanoyl]oxy}-1-azoniabicyclo[2.2.2]octane,
(3R)-1-[2-oxo-2-(pyridin-2-ylamino)ethyl]-3-{[(1-phenylcycloheptyl)carbonyl]oxy}-1-azoniabicyclo[2.2.2]octane,
N-cyclopropyl-3-fluoro-4-methyl-5-{3-[(1-{2-[2-(methylamino)ethoxy]phenyl}cyclopropyl)amino]-2-oxopyrazin-1(2H)-yl}benzamide,
N-cyclohexyl-N-(2-{[2-(5-hydroxy-3-oxo-3,4-dihydro-2H-1,4-benzoxazin-8-yl)ethyl]amino}ethyl)-3-{2-[3-(1-methyl-1H-pyrazol-4-yl)phenyl]ethoxy}propanamide,
N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide,
or a pharmaceutically acceptable salt thereof.

10. A kit comprising a preparation of a first active ingredient which is (1R,3aS,3bS,10aR,10bS,11S,12aS)1-{[(cyanomethyl)sulfanyl]carbonyl}-7-(4-fluorophenyl)-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl furan-2-carboxylate, and a preparation of a second active ingredient selected from a β₂ adrenoreceptor agonist, a dual β₂ adrenoreceptor agonist/M₃ receptor antagonist, a muscarinic antagonist, a p38 kinase inhibitor, a neutrophil elastase inhibitor, a phosphodiesterase PDE4 inhibitor, an IKK2 kinase inhibitor or a non-steroidal glucocorticoid receptor agonist, and instructions for the simultaneous, sequential or separate administration of the preparations to a patient in need thereof.

11. A kit according to claim 10 wherein the second active ingredient is selected from:
N-[2-(diethylamino)ethyl]-N-(2-{[2-(4-hydroxy-2-oxo-2,3-dihydro-1,3-benzothiazol-7-yl)ethyl]amino}ethyl)-3-(2-naphthalen-1-ylethoxy)propanamide,
(3R)-1-[2-(4-fluorophenyl)ethyl]-3-{[(2S)-2-phenyl-2-piperidin-1-ylpropanoyl]oxy}-1-azoniabicyclo[2.2.2]octane,
(3R)-1-[2-oxo-2-(pyridin-2-ylamino)ethyl]-3-{[(1-phenylcycloheptyl)carbonyl]oxy}-1-azoniabicyclo[2.2.2]octane,
N-cyclopropyl-3-fluoro-4-methyl-5-{3-[(1-{2-[2-(methylamino)ethoxy]phenyl}cyclopropyl)amino]-2-oxopyrazin-1(2H)-yl}benzamide,
N-cyclohexyl-N-(2-{[2-(5-hydroxy-3-oxo-3,4-dihydro-2H-1,4-benzoxazin-8-yl)ethyl]amino}ethyl)-3-{2-[3-(1-methyl-1H-pyrazol-4-yl)phenyl]ethoxy}propanamide,
N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide,
or a pharmaceutically acceptable salt thereof.
